(19) United States
(12) Patent Application Publication
(10) Pub. No.: US 2025/0152698 A1
(43) Pub. Date: May 15, 2025

(54) DNA ADJUVANT HYDROGEL-BASED PEPTIDE VACCINE, AND PREPARATION METHOD AND USE THEREOF

(71) Applicants: XIANGFU LABORATORY, Jiaxing (CN); SHANGHAI UNIVERSITY, Shanghai (CN)

(72) Inventors: Lele SUN, Shanghai (CN); Yanfei QU, Shanghai (CN); Yaling ZHANG, Shanghai (CN); Yunlong ZHANG, Shanghai (CN); Qi WANG, Shanghai (CN); Fengyun SHEN, Shanghai (CN); Qinglong YAN, Jiaxing (CN)

(21) Appl. No.: 18/941,518

(22) Filed: Nov. 8, 2024

(30) Foreign Application Priority Data
Nov. 9, 2023 (CN) 202311490433.5

(51) Int. Cl. A61K 39/215; A61K 39/00; A61K 39/39

(52) U.S. Cl. CPC A61K 39/215; A61K 39/39; A61K 2039/55511; A61K 2039/6081; A61K 2039/627

(57) ABSTRACT

A DNA adjuvant hydrogel-based peptide vaccine, and a preparation method and use thereof are provided, belonging to the technical field of biological products. The DNA adjuvant hydrogel-based peptide vaccine includes a peptide-carrier protein, a DNA hydrogel, and a free carrier protein; where the peptide-carrier protein is loaded in the DNA hydrogel or encapsulated in the DNA hydrogel by hybridization; the free carrier protein is encapsulated inside the DNA hydrogel; and the DNA hydrogel is self-assembled from a Y-shaped scaffold and a linker DNA. In the peptide vaccine, the free carrier protein is rapidly released to induce CD4⁺ T cell activation; while the peptide-carrier protein is slowly released, thereby achieving B cell sensitization while activating more CD4⁺ T cells. In this way, B cells are effectively activated to enhance an immune effect of the peptide vaccine.

### CROSS REFERENCE TO RELATED APPLICATION

This patent application claims the benefit and priority of Chinese Patent Application No. 202311490433.5, filed with the China National Intellectual Property Administration on Nov. 9, 2023, the disclosure of which is incorporated by reference herein in its entirety as part of the present application.

### REFERENCE TO SEQUENCE LISTING

A computer readable XML file entitled “GWP20240906489”, that was created on Nov. 1, 2024, with a file size of about 9, 340 bytes, contains the sequence listing for this application, has been filed with this application, and is hereby incorporated by reference in its entirety.

### TECHNICAL FIELD

The present disclosure belongs to the technical field of biological products, and specifically relates to a DNA adjuvant hydrogel-based peptide vaccine, and a preparation method and use thereof.

### BACKGROUND

Peptide vaccines constructed with pathogen antigen epitopes can induce more precise immune responses against pathogenic microorganisms. In addition, the peptide vaccines are prepared by chemical means and have low production costs. Their vaccine composition can be quickly adjusted according to pathogen mutation information, thereby quickly responding to the hazards caused by pathogen mutations. Since the differences in B cell receptors are at the cellular level, a B cell epitope of a pathogen may cause B cell activation in different individuals. However, since the human major histocompatibility complex is responsible for presenting T cell epitopes and there are individual differences, T cell epitopes (including CD4⁺ and CD8⁺ T cell epitopes) that can effectively induce antigen-specific T lymphocyte activation in most human bodies are difficult to be selected from pathogens. Therefore, currently peptide vaccines are mainly constructed using B cell epitopes. However, it is difficult to induce efficient humoral immune responses relying solely on B cell epitopes. This is because the activation of B cells into antibody-secreting cells requires not only multivalent antigens to trigger B cell receptor cross-linking, but also activated TH2-type helper CD4⁺ T cells to provide CD40 ligands for B cells, while B cell epitopes generally cannot induce T cell activation.

At present, in order to make B cell epitopes immunogenic, the B cell epitopes are generally coupled to exogenous carrier proteins (such as OVA, BSA, and KLH) through cross-linking agents, and the T cell epitopes in the carrier protein are used to activate CD4⁺ T cells, thereby inducing effective humoral immunity. However, studies have shown that B cell receptors can generate a first activation signal after receiving antigen stimulation, while it takes several days for T cells to proliferate to a certain quantity after receiving antigen stimulation. If antigen-sensitized B cells do not receive a second stimulation signal from activated CD4⁺ T cells, apoptosis may occur, and these B cells cannot differentiate into plasma cells that produce IgG antibodies. Accordingly, if a large number of CD4⁺ cells can be activated by carrier protein before B cell epitope sensitization on the B cells, it is possible to enhance the immune effect of peptide vaccines. However, this process requires increasing the number of immunizations, thereby increasing vaccination costs and workload.

### SUMMARY

In view of this, a purpose of the present disclosure is to provide a DNA adjuvant hydrogel-based peptide vaccine. In the present disclosure, a free carrier protein is released quickly to induce CD4⁺ T cell activation, and then a carrier protein-B cell epitope conjugate is slowly released, thereby enhancing an immune effect of the peptide vaccine.

The present disclosure provides a DNA adjuvant hydrogel-based peptide vaccine, including a peptide-carrier protein, a DNA hydrogel, and a free carrier protein; where
the peptide-carrier protein is loaded in the DNA hydrogel or encapsulated in the DNA hydrogel by hybridization;
the free carrier protein is encapsulated inside the DNA hydrogel; and
the DNA hydrogel is self-assembled from a Y-shaped scaffold and a linker DNA.

Preferably, the Y-shaped scaffold is assembled from DNA strands having nucleotide sequences shown in SEQ ID NO: 1 to SEQ ID NO: 3; and
the linker DNA is assembled from DNA strands having nucleotide sequences shown in SEQ ID NO: 4 to SEQ ID NO: 5.

Preferably, a quantity of the Y-shaped scaffolds and a quantity of the linker DNAs are at a ratio of 1:1; and
the DNA hydrogel has a molar concentration of 200 μM to 300 μM.

Preferably, a preparation process of the DNA hydrogel includes the following steps:
mixing DNA strands having nucleotide sequences shown in SEQ ID NO: 1 to SEQ ID NO: 3 or DNA strands having nucleotide sequences shown in SEQ ID NO: 4 to SEQ ID NO: 5 at an equal molar ratio in a buffer, and then treating at 95° C. for 15 min to obtain the Y-shaped scaffold and the linker DNA; and
mixing the Y-shaped scaffold and the linker DNA to obtain the DNA hydrogel.

Preferably, a peptide in the peptide-carrier protein includes a B cell epitope polypeptide of a pathogen protein antigen;
preferably, the B cell epitope polypeptide of the pathogen protein antigen includes S3 and S4 of a receptor-binding domain (RBD) of an S protein of a severe acute respiratory syndrome coronavirus 2 (SARS-CoV-2); and
preferably, a carrier protein in the peptide-carrier protein is at least one selected from the group consisting of BSA, OVA, and KLH.

Preferably, a preparation process of the peptide-carrier protein includes the following steps: subjecting a carrier protein BSA to a shaking reaction with dibenzocyclooctyne-N-hydroxysuccinimide (DBCO-NHS), removing unreacted components, and then adding a peptide into an obtained reaction product to allow a freezing reaction; where
the BSA has a molar concentration of 100 μM to 102 μM;
the DBCO-NHS is added at a molar concentration 28 to 32 times the molar concentration of the BSA; and
the peptide is added at a molar concentration 5 to 15 times the molar concentration of the BSA.

Preferably, the peptide-carrier protein is covalently linked to GOD-linker-N₃ and hybridized with DNA strands in the DNA hydrogel; and
the GOD-linker-N₃ has a nucleotide sequence shown in SEQ ID NO: 6.

Preferably, a preparation process of a GOD-linker-N₃-linked peptide-carrier protein includes: adding the GOD-linker-N₃ while adding the peptide at a molar concentration 5 to 15 times the molar concentration of the BSA into the reaction product when the peptide-carrier protein is prepared; where
the GOD-linker-N₃ is added at 1 to 3 times the molar concentration of the BSA.

The present disclosure further provides a preparation method of the DNA adjuvant hydrogel-based peptide vaccine, including a preparation process of a DNA adjuvant hydrogel-based peptide vaccine encapsulating the free carrier protein and the peptide-carrier protein and a preparation process of a DNA adjuvant hydrogel-based peptide vaccine encapsulating the free carrier protein and a hybrid peptide-carrier protein; where
the preparation process of the DNA adjuvant hydrogel-based peptide vaccine encapsulating the free carrier protein and the peptide-carrier protein includes: mixing the free carrier protein and the peptide-carrier protein with the linker DNA, and then mixing an obtained mixture with the Y-shaped scaffold; and
the preparation process of the DNA adjuvant hydrogel-based peptide vaccine encapsulating the free carrier protein and the hybrid peptide-carrier protein includes: mixing the free carrier protein and the GOD-linker-N₃-linked peptide-carrier protein with the linker DNA, and then mixing an obtained mixture with the Y-shaped scaffold.

The present disclosure further provides use of the DNA adjuvant hydrogel-based peptide vaccine or a DNA adjuvant hydrogel-based peptide vaccine prepared by the preparation method in preparation of a vaccine for preventing pathogen infections.

The present disclosure provides a DNA adjuvant hydrogel-based peptide vaccine, including a peptide-carrier protein, a DNA hydrogel, and a free carrier protein; where the peptide-carrier protein is loaded in the DNA hydrogel or encapsulated in the DNA hydrogel by hybridization; the free carrier protein is encapsulated inside the DNA hydrogel; and the DNA hydrogel is self-assembled from a Y-shaped scaffold and a linker DNA. In the present disclosure, the DNA adjuvant hydrogel-based peptide vaccine encapsulates the free carrier protein and the peptide-carrier protein with the DNA hydrogel or couples the peptide-carrier protein by hybridization. After immunization of animals, the free carrier protein is rapidly released to induce CD4⁺ T cell activation; while the peptide-carrier protein is slowly released, thereby achieving B cell sensitization while activating more CD4⁺ T cells. In this way, B cells are effectively activated to enhance an immune effect of the peptide vaccine.

Furthermore, the DNA adjuvant hydrogel-peptide vaccine specifically limits a concentration of the DNA hydrogel. By preparing 3 final concentrations of the DNA hydrogel (250 μM, 500 μM, and 1,000 μM), a fluorescent protein is encapsulated or hybridized to allow small animal imaging experiments, which have proved that the DNA hydrogel at a concentration of 250 μM has the advantage of kinetic differential release.

### BRIEF DESCRIPTION OF THE DRAWINGS

FIG. 1 shows a schematic diagram of the kinetic differential release of components in the DNA adjuvant hydrogel-based peptide vaccine; where

FIGS. 2A-2B show exploration results of an influence of the immunization time interval between carrier protein and peptide vaccine on a humoral immune effect, where FIG. 2A is an exploration timeline of the influence of the immunization time interval between carrier protein and peptide vaccine on the humoral immune effect; FIG. 2B is a specific antibody titer for S4 in mouse serum at different immunization time intervals between carrier protein and peptide vaccine; error bars represent standard errors (n=4, *P<0.1, **P<0.01, ***P<0.001);

FIGS. 3A-3E show coupling results of the BSA with DNA, S3, and S4; where FIG. 3A is the Gelred staining result of BSA-coupled DNA PAGE gel at room temperature; FIG. 3B is the Gelred staining result of BSA-coupled DNA PAGE gel under freezing; FIG. 3C is the Coomassie blue staining result of BSA-coupled DNA PAGE gel under freezing; FIG. 3D is the Coomassie blue staining result of BSA-coupled S3 PAGE gel under freezing; FIG. 3E is the Coomassie blue staining result of BSA-coupled S4 PAGE gel under freezing; M: marker, DNA: DNA control, BSA: BSA control, 5: BSA coupled with 5-fold molar excess of DNA/S3/S4, 10: BSA coupled with 10-fold molar excess of DNA/S3/S4, 15: BSA coupled with 15-fold molar excess of DNA/S3/S4;

FIGS. 4A-4D show construction of hydrogel vaccine experiments and imaging experiment components; where FIG. 4A is the result of Gelred staining PAGE gel of BSA coupled to S3/S4 and DNA; FIG. 4B is the result of Coomassie blue staining PAGE gel of BSA coupled to S3/S4 and DNA; FIG. 4C is the result of Gelred staining PAGE gel of BSA coupled to Alex647-NHS and DNA; FIG. 4D is the result of Coomassie blue staining PAGE gel of BSA coupled to Alex647-NHS and DNA; M: marker, DNA: DNA control, BSA: BSA control, S3: BSA coupled with 13-fold molar excess of S3, S3+DNA: BSA coupled with 13-fold molar excess of S3 and 2-fold molar excess of DNA, S4: BSA coupled with 13-fold molar excess of S4, S4+DNA: BSA coupled with 13-fold molar excess of S4 and 2-fold molar excess of DNA, Alex: BSA coupled with 5-fold molar excess of Alex647-NHS, Alex+DNA: BSA coupled with 5-fold molar excess of Alex647-NHS and 2-fold molar excess of DNA;

FIGS. 5A-5D show change of fluorescence intensity over time when the DNA hydrogels encapsulate or hybridize fluorescent proteins; where FIG. 5A is a bioluminescent image of mice injected with DNA hydrogels hybridized or encapsulated with fluorescent proteins; FIG. 5B is a line chart of the fluorescence intensity over time on the back of mice injected with DNA hydrogels hybridized or encapsulated with fluorescent proteins; FIG. 5C is a line chart of the fluorescence intensity over time on the back of mice injected with different concentrations (125 μM, 250 μM, and 500 μM) of DNA hydrogel-encapsulated fluorescent proteins; FIG. 5D is a line chart of the fluorescence intensity over time on the back of mice injected with different concentrations (125 μM, 250 μM, and 500 μM) of DNA hydrogel-hybridized fluorescent proteins;

FIGS. 6A-6C show antibody content determination results in the serum of mice after immunization with ALG-DNA hydrogel microsphere vaccine; where FIG. 6A is a timeline of the immunization with ALG-DNA hydrogel microsphere vaccine; FIG. 6B is a specific antibody titer for RBD in the serum of mice 14 d after the immunization with ALG-DNA hydrogel microsphere vaccine; FIG. 6C is a specific antibody titer for RBD in the serum of mice 21 d after the immunization with ALG-DNA hydrogel microsphere vaccine; error bars represent standard deviation (n=4, *P<0.1, **P<0.01);

FIGS. 7A-7B show activation of Th2 CD4⁺ T cells 7 d after immunization with ALG-DNA hydrogel microsphere vaccine; where FIG. 7A is a statistical bar graph of the activation results of Th2 CD4⁺ T cells 7 d after immunization; FIG. 7B is a flow cytometric graph of the proliferation of Th2 CD4⁺ T cells 7 d after immunization; error bars represent standard deviations (n=4, **P<0.01, ****P<0.0001);

FIGS. 8A-8B show activation of Th2 CD4⁺ T cells 14 d after immunization with ALG-DNA hydrogel microsphere vaccine; where FIG. 8A is a statistical bar graph of the activation results of Th2 CD4⁺ T cells 14 d after immunization; FIG. 8B is a flow cytometric graph of the proliferation of Th2 CD4⁺ T cells 14 d after immunization; error bars represent standard deviations (n=4, *P<0.1, **P<0.01, ***P<0.001, ****P<0.0001); and

FIGS. 9A-9B show evaluation results of a biosafety of polypeptide vaccines based on the kinetic differential release of ALG-DNA hydrogel microspheres; where FIG. 9A is a line chart of the weight changes of mice after immunization with ALG-DNA hydrogel microspheres; FIG. 9B is the H&E staining sections of the main organs of mice in an ALG-DNA hydrogel vaccine immunization group, a two-dose free vaccine immunization group, and a blank group; scale bar: 100 μm.

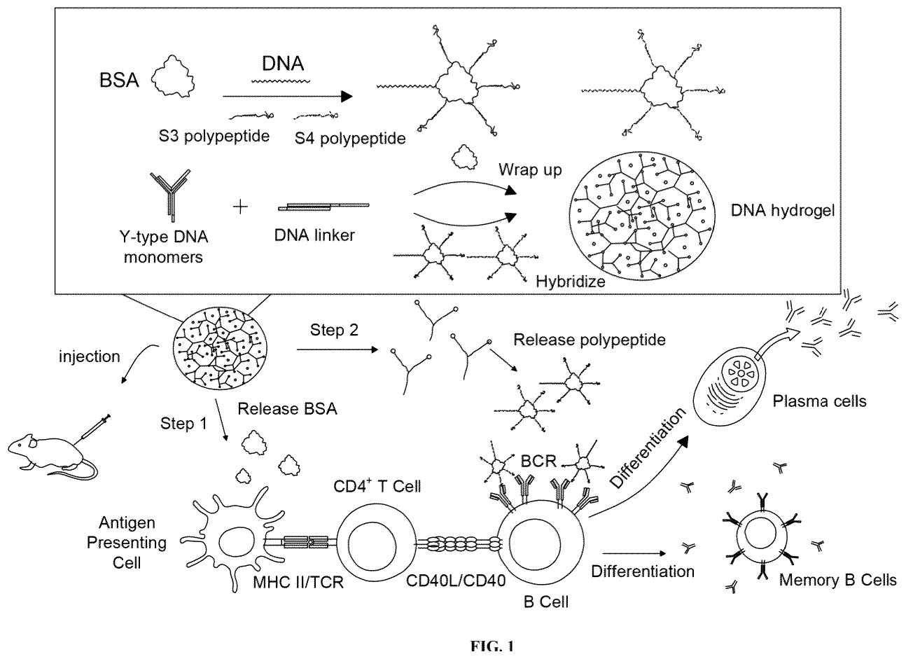

### DETAILED DESCRIPTION OF THE EMBODIMENTS

The present disclosure provides a DNA adjuvant hydrogel-based peptide vaccine, including a peptide-carrier protein, a DNA hydrogel, and a free carrier protein; where
the peptide-carrier protein is loaded in the DNA hydrogel or encapsulated in the DNA hydrogel by hybridization;
the free carrier protein is encapsulated inside the DNA hydrogel; and
the DNA hydrogel is self-assembled from a Y-shaped scaffold and a linker DNA.

In the present disclosure, the Y-shaped scaffold is preferably assembled from DNA strands having nucleotide sequences shown in SEQ ID NO: 1 to SEQ ID NO: 3. The assembly is preferably conducted at 95° C. for 15 min. The DNA strands shown in SEQ ID NO: 1 to SEQ ID NO: 3 are at a molar ratio of preferably 1:1:1. These DNA strands contain fully thiolated linear CpG ODNs that have strong immunostimulatory activity against B cells. The linker DNA is assembled from DNA strands having nucleotide sequences shown in SEQ ID NO: 4 to SEQ ID NO: 5. These DNA strands contain class B CpG ODN specific for murine TLR9. The assembly is preferably conducted at 95° C. for 15 min. The DNA strands shown in SEQ ID NO: 4 to SEQ ID NO: 5 are at a molar ratio of preferably 1:1. The names of the DNA strands used to construct the DNA hydrogel are shown in Tables 1 to 3. The principle of sequence assembly of the Y-shaped scaffold is that the bases 9 to 20 in 2006-1-S are complementary to the bases 21 to 32 in 2006-3; the bases 21 to 32 in 2006-1-S are complementary to the bases 9 to 20 in 2006-2; the bases 21 to 32 in 2006-2 are complementary to the bases 9 to 20 in 2006-3. The principle of the assembly of the linker DNA sequence and the Y-shaped scaffold is that the bases 22 to 29 in 1826-1-linker-S and the bases 1 to 8 in 1826-2 are the same, and are complementary to the bases 1 to 8 of the three DNAs forming the Y-shaped scaffold. The bases 30 to 49 in 1826-1-linker-S are complementary to the bases 9 to 28 in 1826-2. The GOD linker-N₃ is complementary to bases 1 to 20 in 1826-1-linker-S and bases 29 to 48 in 1826-2. Modification of phosphorothioate diester bonds in each DNA strand can improve the stability of DNA in physiological environments. The DNA hydrogel has a concentration of preferably 200 μM to 300 μM, more preferably 230 μM to 280 μM, and most preferably 250 μM. Experiments have showed that 250 μM DNA hydrogel has a better sustained release effect than that of other concentrations (125 μM, 500 μM), such that subsequent experiments are conducted using the 250 μM DNA hydrogel.

In the present disclosure, a preparation process of the DNA hydrogel preferably includes the following steps: mixing the Y-shaped scaffold and the linker DNA at an equal molar ratio to obtain the DNA hydrogel. A quantity of the Y-shaped scaffolds and a quantity of the linker DNAs are at a ratio of preferably 1:1. The mixing is conducted at preferably 20° C. to 28° C., more preferably 23° C. to 26° C., and even more preferably 25° C.

In the present disclosure, a peptide in the peptide-carrier protein preferably includes a B cell epitope polypeptide of a pathogen protein antigen. There is no particular limitation on a type of the pathogen, and any pathogen known in the art may be used, such as bacteria, viruses, fungi, mycoplasmas, and chlamydia. The B cell epitope polypeptide of the pathogen protein antigen preferably includes S3 and S4 of a receptor-binding domain (RBD) of an S protein of SARS-CoV-2. The S3 has an amino acid sequence shown in SEQ ID NO: 7 (GDEVRQIAP); the S4 has an amino acid sequence shown in SEQ ID NO: 8 (NLDSKV). The B cell epitope polypeptide is preferably a polypeptide with an azide group (N₃) modified at the end; the polypeptide undergoes addition between the azide group and the alkyne group in a DBCO group, such that BSA is coupled to the polypeptide. Experiments have shown that a hysteresis effect of the coupling of BSA and S4-N₃ is not as significant as that of the coupling of BSA and S3-N₃, which may be because a molecular weight of S3-N₃ is larger than that of S4-N₃.

In the present disclosure, a carrier protein in the peptide-carrier protein is preferably at least one selected from the group consisting of BSA, OVA, and KLH. In the examples, BSA is used as an example to illustrate the immune effect of the peptide vaccine, but this should not be construed as a limitation to the protection of the present disclosure.

In the present disclosure, a preparation process of the peptide-carrier protein preferably includes the following steps: subjecting a carrier protein BSA to a shaking reaction with DBCO-NHS, removing unreacted components, and then adding a peptide into an obtained reaction product to allow a freezing reaction. The BSA has a concentration of preferably 100 μM to 102 μM, more preferably 101 μM. The DBCO-NHS Desirable is added at preferably 28 to 32 times, more preferably 30 times the molar concentration of the BSA. The peptide is added at preferably 5 to 15 times, more preferably 13 times the molar concentration of the BSA. The freezing reaction is conducted at preferably −18° C. to −22° C., more preferably −20° C. The freezing reaction is conducted for preferably 10 h to 14 h, more preferably 12 h.

In the present disclosure, preferably, the peptide-carrier protein is covalently linked to GOD-linker-N₃ and hybridized with DNA strands in the DNA hydrogel. The GOD-linker-N₃ has a nucleotide sequence shown in SEQ ID NO: 6. A preparation process of a GOD-linker-N₃-linked peptide-carrier protein preferably includes: adding the GOD-linker-N₃ while adding the peptide at a molar concentration 5 to 15 times the molar concentration of the BSA into the reaction product when the peptide-carrier protein is prepared. The GOD-linker-N₃ is added at preferably 1 to 3 times, more preferably 2 times the molar concentration of the BSA.

The present disclosure further provides a preparation method of the DNA adjuvant hydrogel-based peptide vaccine, including a preparation process of a DNA adjuvant hydrogel-based peptide vaccine encapsulating the free carrier protein and the peptide-carrier protein and a preparation process of a DNA adjuvant hydrogel-based peptide vaccine encapsulating the free carrier protein and a hybrid peptide-carrier protein; where
the preparation process of the DNA adjuvant hydrogel-based peptide vaccine encapsulating the free carrier protein and the peptide-carrier protein includes: mixing the free carrier protein and the peptide-carrier protein with the linker DNA, and then mixing an obtained mixture with the Y-shaped scaffold; and
the preparation process of the DNA adjuvant hydrogel-based peptide vaccine encapsulating the free carrier protein and the hybrid peptide-carrier protein includes: mixing the free carrier protein and the GOD-linker-N₃-linked peptide-carrier protein with the linker DNA, and then mixing an obtained mixture with the Y-shaped scaffold.

In the present disclosure, the free carrier protein, the peptide-carrier protein, the linker DNA, and the Y-shaped scaffold are at a molar ratio of preferably (0.4-0.6):(0.4-0.6):(0.8-1.2):(0.8-1.2), more preferably 0.5:0.5:1:1. The mixing is conducted at preferably 18° C. to 27° C., more preferably 20° C. to 25° C., and even more preferably 23° C.

The present disclosure further provides use of the DNA adjuvant hydrogel-based peptide vaccine or a DNA adjuvant hydrogel-based peptide vaccine prepared by the preparation method in preparation of a vaccine for preventing pathogen infections.

In the examples of the present disclosure, an encapsulated group (BSA&BSA-AgP@gel) has the lowest antibody titer among all immunization groups, and is significantly different from the other groups, indicating that the rapid release of vaccine components cannot produce a desirable immune effect. An experimental group (BSA@BSA-AgP-gel) produces the highest antibody titer, which is significantly different from that of a full hybrid group ((BSA&BSA-AgP)-gel), indicating that the kinetic differential release vaccine that rapidly releases the free carrier protein to activate T cells and then slowly releases the peptide vaccine to activate B cells has a better immune effect than that of the vaccine with slow release of all vaccine components. The experimental group (BSA@BSA-AgP-gel) also has significant differences from the two-dose group (Free BSA&BSA-AgP), indicating that the humoral immune effect of the kinetic differential release vaccine is better than that produced by the vaccine with two immunizations, which means that the kinetic differential release vaccine can reduce the number of immunizations.

The experimental group (BSA@BSA-AgP-gel) still maintains a relatively high antibody titer level, indicating that the kinetic differential release vaccine has a relatively long-lasting immune effect. The antibody titer levels in other treatment groups are slightly lower than 14 d after immunization. The mice in experimental group (BSA@BSA-AgP-gel) have the highest antibody titer 14 d and 21 d after immunization in each group, indicating that the experimental group produces the best humoral immune effect. There is no significant difference between the full hybrid group ((BSA&BSA-AgP)-gel) and the encapsulated group (BSA&BSA-AgP@gel). This may be because all vaccine components in the encapsulated group (BSA&BSA-AgP@gel) are quickly released at this time, while some vaccine components in the full hybrid group ((BSA&BSA-AgP)-gel) have not yet been released. The experimental group (BSA@BSA-AgP-gel) has the highest Th2 CD4⁺ T cell activation effect, which is significantly different from that of all other groups, indicating that the kinetic differential release vaccine has a better T cell activation effect than that of the hydrogel immunization groups.

The DNA adjuvant hydrogel-based peptide vaccine, and the preparation method and the use thereof as provided by the present disclosure will be described in detail in connection with the following examples, but they should not be construed as limiting the claimed scope of the present disclosure.

In the examples of the present disclosure, some of the reagents, instruments, and experimental animals involved are from the following sources:

Experimental reagents: all DNA primers are synthesized and purified by Sangon Biotech Co., Ltd. Bovine serum albumin (BSA) is purchased from Sigma-Aldrich. Alex647-NHS is purchased from Thermo Fisher. S3-N₃, S4-N₃, OVA-S4, and OVA-S3 are purchased from GenScript. Isoflurane is purchased from RWD Life Science, Shenzhen. DBCO-NHS is purchased from MedChemExpress. PBST buffer, 30% Acr-Bis, TAE buffer, 10% Aps, TEMED, loading buffer, acetic acid, anhydrous ethanol, no protein blocking solution, TMB, and hematoxylin & eosin dye are purchased from Sangon Biotech Co., Ltd. Horseradish peroxidase (HRP)-conjugated goat anti-mouse IgG, 4% paraformaldehyde, and immunostaining permeabilization solution are purchased from Beyotime. Sodium chloride, calcium chloride, and sodium alginate are purchased from Sinopharm Chemical Reagent Co., Ltd. Flow cytometry antibodies: FITC anti-mouse CD3F, PerCP anti-mouse CD4, and PE/Cyanine7 anti-mouse IL-4 are purchased from BioLegend.

The experimental instruments include: metal bath (MDB100-D, JOANLAB Equipment CO., LTD.). Digital multifunctional mixer (VM-500Pro, JOANLAB Equipment CO., LTD.). Gel imaging system (SH-510, Hangzhou Shenhua Science Technology Co., Ltd.). Electronic analytical balance (FA124C, Shanghai Lichen Bangxi Instrument Technology Co., Ltd.). Quantitative electronic scale (MT01, Shenzhen Meifu Electronics Co., Ltd.). Syringe pump (ZS100-01, Changzhou Enpei Instrument Manufacturing Co., Ltd.). Electrophoresis apparatus (EPS-300, Shanghai Tanon Life Science Co., Ltd.). Confocal microscope (Zeiss, Germany). Fluorescence spectrometer (F-7000, HITACHI, Japan). Three-dimensional Small Animal Imaging System (PerkinElmer, Austria). Multifunctional microplate reader (VICTOR X3, PerkinElmer, USA). Flow cytometer (MoFlo XDP, Beckman, UK).

Experimental animals include: female C57BL/6 mice (6-8 weeks old). Female BALB-c mice (6-8 weeks old). The use of experimental animals is approved by the Experimental Animal Ethics Committee of Shanghai University.

### Example 1

A preparation method of a DNA adjuvant hydrogel-based peptide vaccine included the following steps:
1. Preparation of fluorescent dye-modified BSA: 101 μM BSA was added with 5-fold molar excess of fluorescent Alex647-NHS, reacted at room temperature for 2 h, and ultrafiltered with a 30 k ultrafiltration tube until the liquid under ultrafiltration had no fluorescence to obtain the fluorescent dye-modified BSA.
2. Preparation of fluorescent dye-modified BSA-coupled DNA: 10-fold molar excess DBCO-NHS was added to 101 μM BSA, and then 5-fold excess fluorescent Alex647-NHS was added, reacted at room temperature for 2 h, and ultrafiltered using a 30 k ultrafiltration tube until the liquid under ultrafiltration had no fluorescence. 2-fold molar excess of GOD-linker-N₃ (Table 1) was added to the reaction system, and a reaction was conducted under freezing overnight to obtain the fluorescent dye-modified BSA-DNA.
3. Preparation of BSA-coupled DNA: 101 μM BSA was reacted with 10-fold molar excess DBCO-NHS at room temperature for 1 h under shaking, ultrafiltered 3 times with a 30 k ultrafiltration tube, and 2-fold molar excess GOD-linker-N₃ was added and frozen overnight to obtain BSA-DNA.
4. Preparation of BSA-coupled polypeptides: 101 μM BSA was reacted with 30-fold molar excess DBCO-NHS at room temperature for 1 h under shaking, and ultrafiltered 3 times with a 30 k ultrafiltration tube, and 13-fold molar excess polypeptides (S3-N₃ or S4-N₃) were added and frozen overnight to obtain BSA-S3 and BSA-S4.
5. Construction of BSA-coupled DNA and polypeptides: 101 μM BSA was reacted with 30-fold molar excess DBCO-NHS at room temperature for 1 h under shaking, ultrafiltered 3 times with a 30 k ultrafiltration tube, and 2-fold molar excess GOD-linker-N₃ and 13-fold molar excess polypeptide (S3-N₃ or S4-N₃) were added and frozen overnight to obtain BSA-DNA-S₃ and BSA-DNA-S₄.
6. DNA hydrogel was formed by self-assembly and cross-linking of Y-shaped scaffold structure and linker structure. The Y-shaped scaffold sequence contained a fully thiolated linear CpG ODN, which had a strong immunostimulatory activity on B cells; the linker sequence contained a class B CpG ODN specific to mouse TLR9. The DNA strands of the designed sequences used in the Y-shaped scaffold structure or linker structure (Table 2 and Table 3, where the 2,006 component contained a fully thiolated linear CpG ODN, which had strong immunostimulatory activity on B cells; and the 1,826 component contained a class B CpG ODN specific for mouse TLR9) were mixed at an equimolar ratio in a buffer (100 mM Hepes, 100 mM Hepes-Na, 1 M NaCl, pH=7.5), heated in a 95° C. metal bath for 15 min, and cooled to room temperature to form the Y-shaped scaffold structure and the linker structure. The Y-shaped scaffold structure and the linker structure were uniformly mixed at a volume ratio of 1:1 at room temperature to form a 250 μM DNA hydrogel.

TABLE 1
GOD-linker-N₃ sequence

| Name | Sequence (5′-3′) |
|---|---|
| GOD-linker-N₃ | G*G*A*G*C*T*G*T*G*T*T*A*C*C*A* |
| | A*G*C*A*C (3′ Azide(N₃)) |
| | (SEQ ID NO: 6) |

TABLE 2
Y-shaped scaffold sequence of DNA hydrogel

| Name | Sequence (5′-3′) |
|---|---|
| 2006-1-S | CGATTGACT*C*G*T*C*G*T*T*T*T*G*T* |
| | C*G*T*T*T*T*G*T*C*G*T*T |
| | (SEQ ID NO: 1) |
| 2006-2: | CGATTGACAACGACAAAACGCACGCTGTCCTA |
| | (SEQ ID NO: 2) |
| 2006-3: | CGATTGACTAGGACAGCGTGACAAAACGACGA |
| | (SEQ ID NO: 3) |

TABLE 3
linker sequence of DNA hydrogel

| Name | Sequence (5′-3′) |
|---|---|
| 1826-1- | G*T*G*C*T*T*G*G*T*A*A*C*A*C* |
| linker-S | A*G*C*T*C*CTGTCAATCGT*C*C*A* |
| | T*G*A*C*G*T*T*C*C*T*G*A*C*G* |
| | T*T (SEQ ID NO: 4) |
| 1826-2 | GTCAATCGAACGTCAGGAACGTCATGGA |
| | (SEQ ID NO: 5) |

NOTE: * indicated that the phosphorothioate diester bond was modified between two bases.

### Example 2

### Influence of the Time Interval Between Carrier Protein and Peptide Vaccine on Humoral Immunity

In order to determine the effect of the immunization time interval between carrier protein and peptide vaccine on the humoral immune effect of peptide vaccine, experiments with different immunization time intervals were set up to measure the antibody titer in the serum of each group of mice. 20 female C57 mice (20 μg body weight) were randomly divided into 5 groups, one of which was not vaccinated as a blank control.

A first group (Group 1) was injected subcutaneously on the back of mice with 200 μg BSA, 10 μg CpG, and 10 μg BSA-S4 on day 0, and then injected with 10 μg BSA-S4 again after an interval of 7 d.

A second group (Group 2) was injected subcutaneously with 200 μg BSA and 10 μg CpG on the back of mice on day 0, and then injected with 10 μg BSA-S4 after 3 d, and then injected with 10 μg BSA-S4 again after 7 d.

A third group (Group 3) was injected subcutaneously with 200 μg BSA and 10 μg CpG on the back of mice on day 0, and then injected with 10 μg BSA-S4 after 6 d, and then injected with 10 μg BSA-S4 again after 7 d.

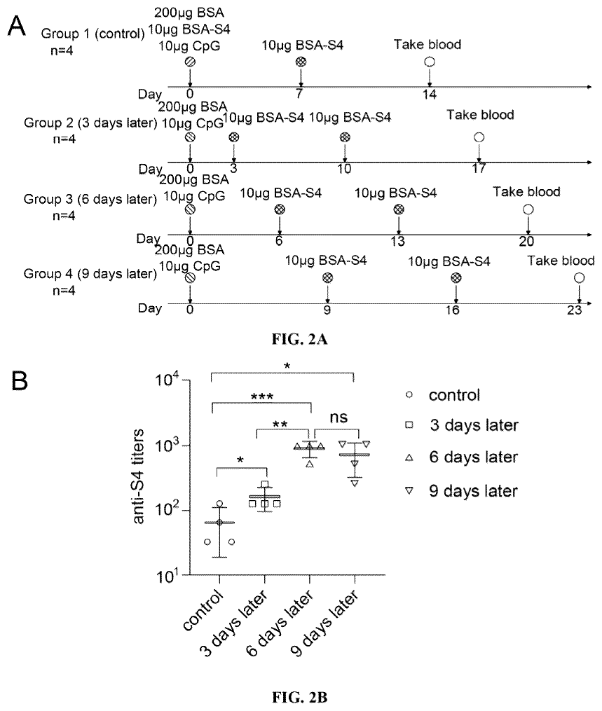

A fourth group (Group 4) was injected subcutaneously with 200 μg BSA and 10 μg CpG on the back of mice on day 0, and then injected with 10 μg BSA-S4 after 9 d, and then injected with 10 μg BSA-S4 again after 7 d. Blood samples were collected every 7 d after the last immunization in each group, and the immunization timeline was shown in FIG. 2A.

For the determination of specific antibody titers in mouse sera after vaccination, 2 μg/100 μL OVA-S4 was coated on high-absorption 96-well Costar plates and incubated overnight at 4° C. Excess OVA-S4 was discarded and the samples were rinsed 2 to 3 times with 1×PBST. The samples were blocked with 200 μL of protein-free blocking buffer at room temperature for 1 h and then rinsed 3 times with 1×PBST. Serum samples with different dilution multiples (1:23 to 1:210) were added to the plate and incubated at room temperature for 2 h, and then rinsed 3 to 4 times with 1×PBST. 100 μL of horseradish peroxidase (HRP)-conjugated goat anti-mouse IgG (1:1000) was added and incubated at room temperature for 30 min to 60 min, and then rinsed 5 times with 1×PBST. Finally, 100 μL of TMB substrate was added for color development, and the absorbance values at 450 nm and 620 nm were detected using an enzyme reader. The specific antibody titer of each group of mice against SARS-CoV-2 polypeptide S4 was measured. The antibody titer of each immunization group was measured by comparison with the blank group, that is, a signal was considered detectable if the absorbance value was greater than twice the average value of the blank group absorbance.

Blood was collected one week after the last immunization to measure specific antibody titers, and antibody titers of the sera of mice in each experimental group against S4 were shown in FIG. 2B. The results showed that the injection of BSA-S4 3, 6, and 9 d after BSA immunization was significantly different from the injection of BSA and BSA-S4 on the same day, indicating that injecting carrier protein to activate T cells and then injecting peptide vaccine to activate B cells had a better humoral immune effect. The mice injected with BSA-S4 6 d after BSA had the highest antibody titer, and the antibody titer of the control group injected with BSA and BSA-S4 on the same day had the highest significant difference. In addition, the antibody titer of mice injected with BSA-S4 6 d after BSA immunization was significantly different from that of mice injected with BSA-S4 3 d after BSA immunization, indicating that the humoral immunity effect of BSA-S4 injected 6 d after BSA immunization was the best.

### Example 3

In order to explore the optimal conditions for the coupling reaction of BSA with DNA and polypeptides, the coupling reaction of BSA modified with DBCO-NHS and GOD-linker-N₃ was conducted at room temperature and under freezing conditions, and the coupling efficiency was determined by comparing the hysteresis relative to the control.

BSA-DNA coupling at room temperature: 101 μM BSA was reacted with 30-fold molar excess DBCO-NHS at room temperature for 1 h under shaking, ultrafiltered 3 times with a 30 k ultrafiltration tube, and 5-fold, 10-fold, and 15-fold molar excess GOD-linker-N₃ were added, and the reaction was allowed to stand at room temperature overnight.

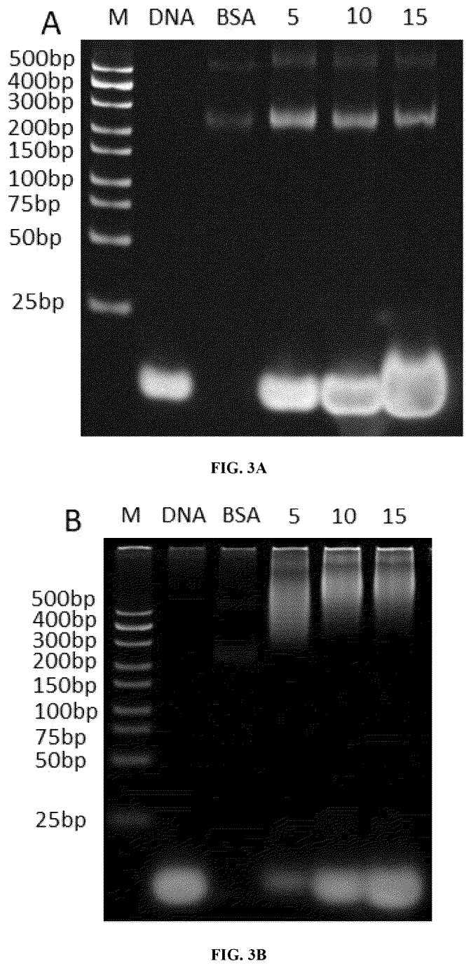

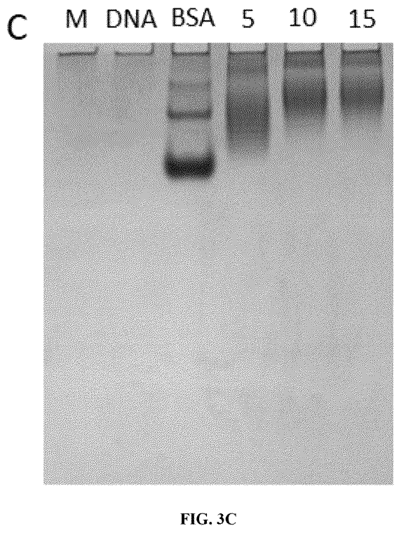

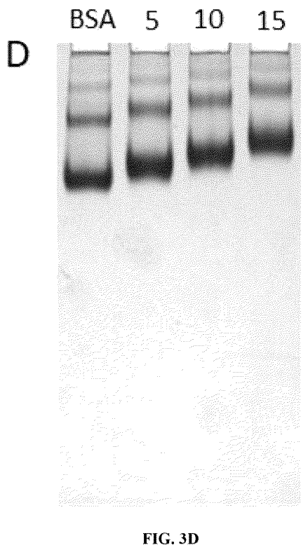

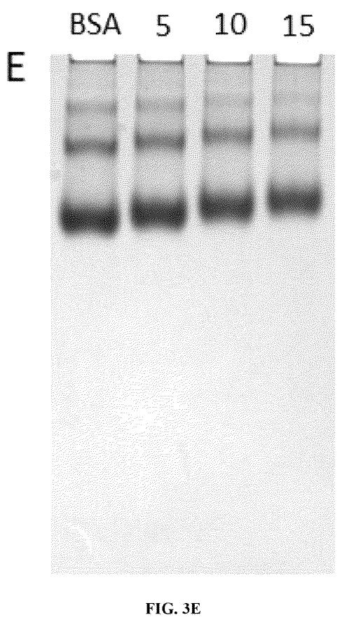

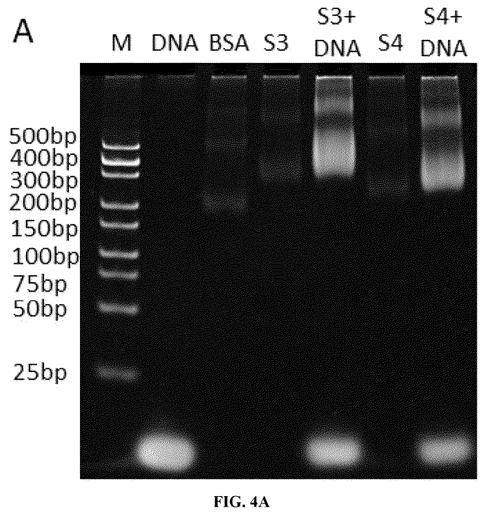

As shown in FIG. 3A, although the BSA coupled with GOD-linker-N₃ at room temperature had a DNA band at the corresponding position, it did not lag significantly compared with the BSA control modified with DBCO-NHS. Moreover, as the amount of GOD-linker-N₃ input increased, the connection efficiency did not increase, indicating that there was a poor coupling effect of the room-temperature reaction.

Since BSA coupling to DNA was not effective at room temperature, the coupling reaction temperature was optimized, specifically the DBCO-NHS-modified BSA and GOD-linker-N₃ were frozen and reacted overnight. BSA-DNA coupling at low temperature: 101 μM BSA was reacted with 30-fold molar excess DBCO-NHS at room temperature for 1 h under shaking, ultrafiltered 3 times with a 30 k ultrafiltration tube, and 5-fold, 10-fold, and 15-fold molar excess GOD-linker-N₃ were added, and the reaction was conducted under freezing at −20° C. overnight. FIG. 3B was the result of Gelred dye staining, and FIG. 3C was the result of Coomassie brilliant blue staining. Compared with the BSA control modified with DBCO-NHS, the BSA coupled with GOD-linker-N₃ produced an overall obvious hysteresis and a step-like band, indicating that BSA was successfully coupled with different proportions of GOD-linker-N₃. As the input amount of GOD-linker-N₃ increased, the lag effect became more obvious. Compared with FIG. 3A, it was be seen that the coupling effect between BSA modified with DBCO-NHS and GOD-linker-N₃ was better under freezing conditions, such that the subsequent coupling experiments were conducted at low temperature under freezing conditions.

BSA-coupled peptide (BSA-AgP) at low temperature: 101 μM BSA was reacted with 30-fold molar excess DBCO-NHS at room temperature for 1 h by shaking, ultrafiltered 3 times with a 30 k ultrafiltration tube, and 5-fold, 10-fold, or 15-fold molar excess S3-N₃ or S4-N₃ were added, and the reaction was conducted under freezing at −20° C. overnight. FIG. 3D was the coupling result of BSA and S3-N₃, and FIG. 3E was the coupling result of BSA and S4-N₃. It was seen that the band was obviously lagged, indicating that BSA and the polypeptide were successfully coupled. Moreover, the band hysteresis became more obvious as the excess of peptide increased, indicating that BSA could be successfully coupled to 15-fold excess of polypeptide. However, a hysteresis effect of the coupling of BSA and S4-N₃ was not as significant as that of the coupling of BSA and S3-N₃, which might be because a molecular weight of S3-N₃ was larger than that of S4-N₃.

### Example 4

### Methods for Constructing Components of Hydrogel Vaccine Experiments and Imaging Experiments

Vaccine components and samples required for fluorescence imaging were prepared under conditions of overnight freezing coupling. From the results in 2, it was seen that BSA could still produce band hysteresis when reacting with 15-fold molar excess of DNA or polypeptide. In order to attach as many peptides as possible to BSA as multivalent B cell epitopes, 15-fold molar excess of components were added to react with BSA in the preparation of vaccine components. In order to make the BSA with polypeptides hybridize to the DNA hydrogel backbone, it was necessary to couple the DNA (GoD-linker-N₃) that was complementary to the DNA hydrogel backbone to the BSA. Therefore, a 13-fold molar excess of polypeptides (S3/S4) and a 2-fold molar excess of GoD-linker-N₃ were coupled to the BSA. BSA-AgP linked with GoD-linker-N₃ was obtained.

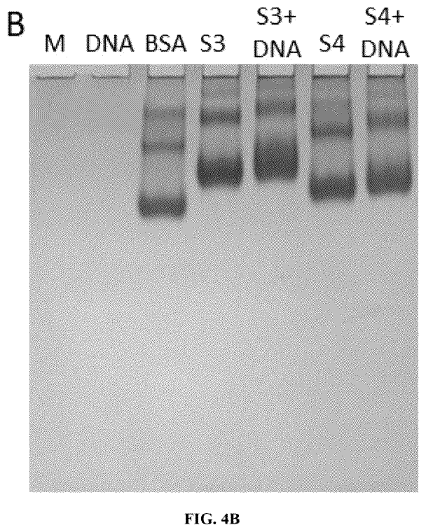

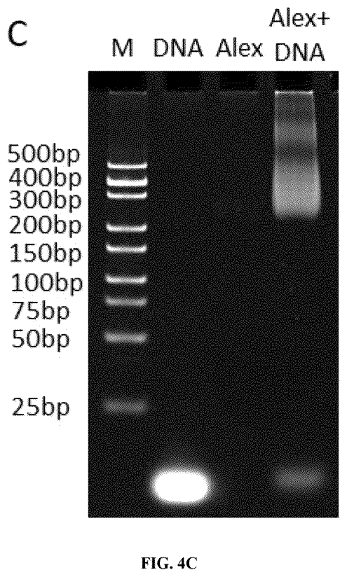

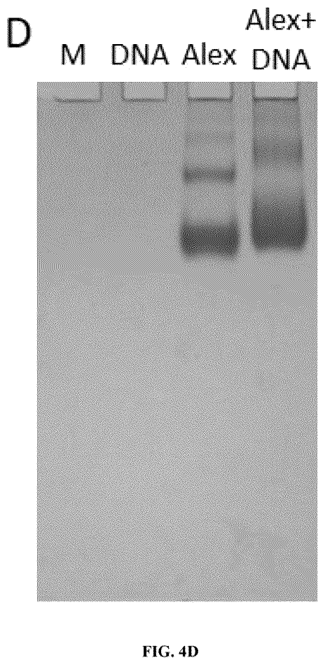

FIG. 4A and FIG. 4B are were results of Gelred nucleic acid staining and Coomassie brilliant blue protein staining, respectively. The gel electrophoresis results showed that when BSA was coupled with 13-fold excess S3 or S4, the overall band produced obvious hysteresis, and the degree of S3 hysteresis was stronger than that of S4, which was consistent with the results in Example 3 and was due to the fact that the molecular weight of S3 was larger than that of S4. When BSA was reacted with 13-fold molar excess of S3 or S4 and then with 2-fold molar excess of GoD-linker-N₃, it was observed that the band was further lagged overall, indicating that GoD-linker-N₃ was also successfully coupled with BSA.

Next, BSA modified with fluorescent dye and GoD-linker-N₃ was prepared for imaging experiments. FIG. 4C and FIG. 4D were the results of Gelred nucleic acid staining and Coomassie brilliant blue protein staining, respectively. From left to right in the figure there were DNA marker, DNA control, BSA control modified with DBCO-NHS and 5-fold molar excess of fluorescent Alex647-NHS, and BSA linked to 5-fold molar excess of Alex647-NHS and 2-fold molar excess of GoD-linker-N₃. The BSA band coupled with GoD-linker-N₃ was overall lagged, indicating that GoD-linker-N₃ was coupled to BSA.

### Example 5

### Preparation of DNA Hydrogel

The DNA hydrogel was formed by self-assembly and cross-linking of a Y-shaped scaffold structure and a linker structure. The DNA strands of the designed sequences used for the two structures were mixed at an equimolar ratio in a buffer solution (10 mM Hepes, 10 mM Hepes-Na, 100 mM NaCl, pH=7.5), heated in a 95° C. metal bath for 10 min, and cooled to room temperature to form the Y-shaped scaffold structure and linker structure. The Y-shaped scaffold structure and the linker structure were uniformly mixed at a volume ratio of 1:1 at room temperature to form DNA hydrogels with final concentrations of 125 μM, 250 μM, and 500 μM.

Preparation of DNA hydrogel-encapsulated fluorescent BSA: 100 pmol of fluorescently modified BSA was added to the linker structure component of the DNA hydrogel, and then the Y-shaped scaffold structure and the linker structure were mixed at a volume ratio of 1:1 to form L of DNA hydrogels with final concentrations of 125 μM, 250 μM, and 500 μM.

Preparation of DNA hydrogel-hybridized fluorescent BSA: 100 pmol of fluorescently modified BSA linked with GOD-linker-N₃ was added to the linker structure component of DNA hydrogel and mixed thoroughly, and the Y-shaped scaffold structure and the linker structure were mixed at a volume ratio of 1:1 to form 25 μL of DNA hydrogels with final concentrations of 125 μM, 250 μM, and 500 μM.

### Example 6

### Characterization of the Kinetic Differential Release of DNA Hydrogels In Vivo

Determination of release rate of DNA hydrogel encapsulated or hybridized fluorescent BSA: DNA hydrogels with 3 final concentrations (250 μM, 500 μM, and 1,000 μM) were prepared according to Example 5 to encapsulated fluorescent protein and hybridized fluorescent protein, respectively. The hydrogel was injected subcutaneously into the back of BALB-c mice for in vivo small animal imaging, and imaging was conducted every 24 h until the fluorescence intensity dropped below 10% of the fluorescence intensity on the first day.

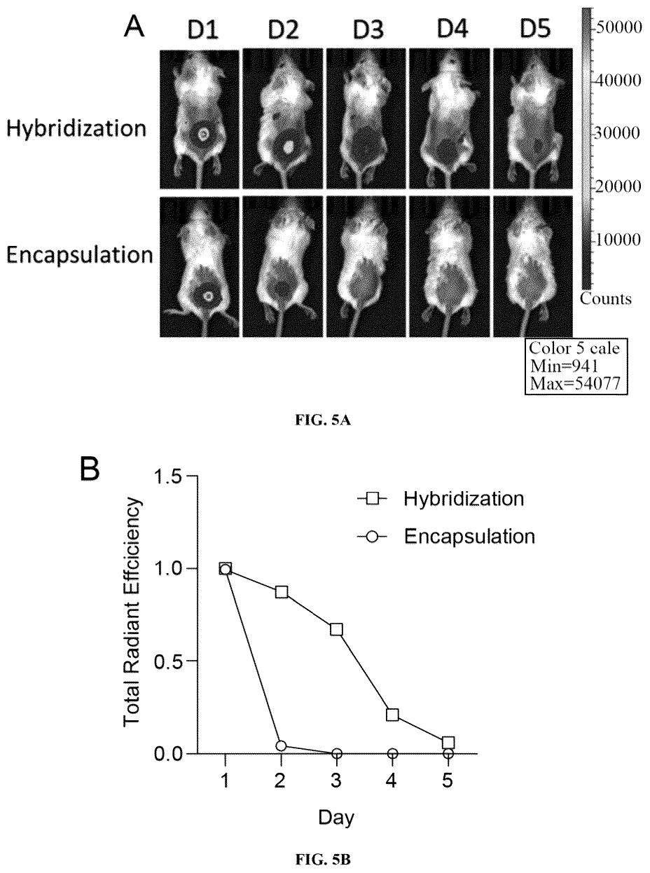

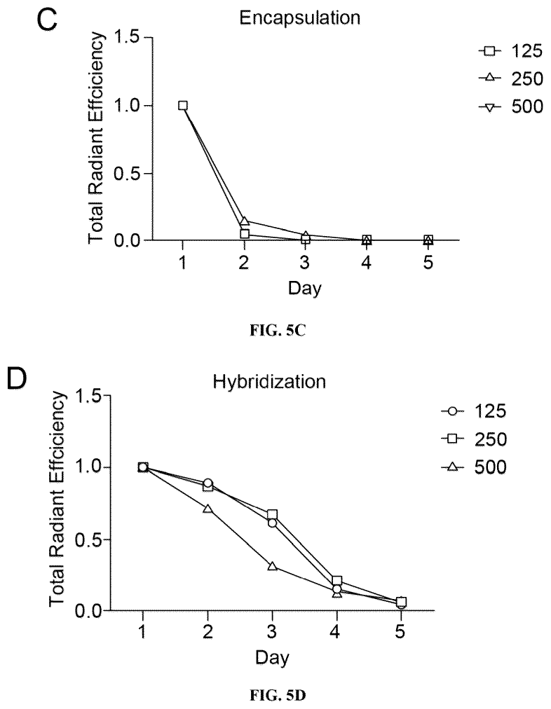

The experimental results were shown in FIG. 5A and FIG. 5B. According to the ratio of the fluorescence intensity value on each day to the fluorescence intensity value on the first day, it was seen that the fluorescence intensity of the encapsulated group on the second day had dropped to 15% of that on the first day; while the fluorescence intensity of the hybridized group on the second day was still 87% of that on the first day, and on the third day it was still 67% of that on the first day, and the fluorescence was completely released till day five. This indicated that DNA hydrogel-hybridized BSA could play a role in sustained release of BSA. For DNA hydrogels of different concentrations, as shown in FIG. 5C and FIG. 5D, the 250 μM DNA hydrogel had a better sustained release effect than other concentrations, such that the subsequent experiments were conducted using 250 μM DNA hydrogel.

### Example 7

### Determination of the Effect of Kinetic Differential Release of DNA Hydrogel on Humoral Immunity of Peptide Vaccine

The main functional components of the DNA hydrogel-based kinetic differential release vaccine were composed of free carrier protein and peptide vaccine. The vaccine components were linked to the hydrogel scaffold by coupling GOD-linker-N₃ on the carrier protein, thereby achieving kinetic differential release.

The construction methods of BSA-coupled DNA, BSA-coupled polypeptide, and BSA-coupled DNA and polypeptide were shown in Example 1. According to the different types of 250 μM DNA hydrogels prepared in Example 6, polypeptide vaccines were constructed for immunizing mice.

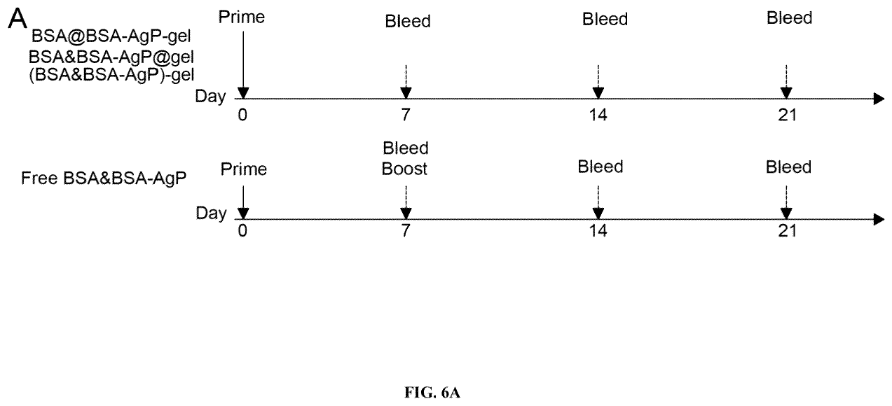

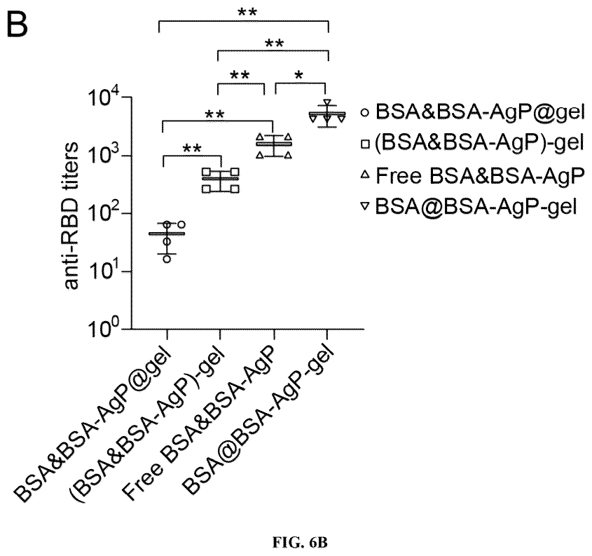

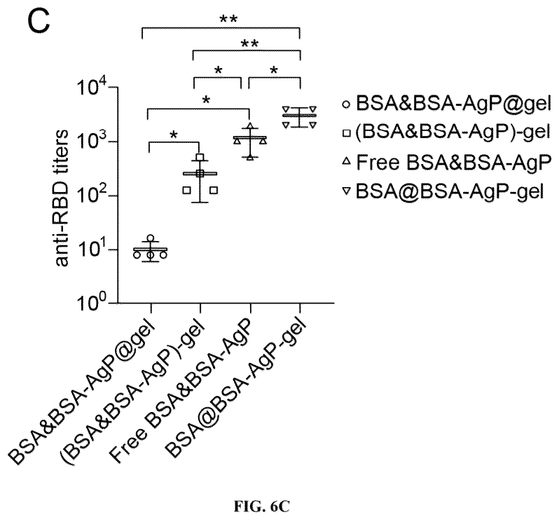

A preparation method of the BSA@BSA-AgP-gel polypeptide vaccine included: BSA was mixed with the BSA-AgP linked to GOD-linker-N₃ prepared in Example 4 and the linker DNA, and then a mixed system was mixed with a Y-shaped scaffold, where the BSA, BSA-AgP, linker DNA, and Y-shaped scaffold were at a molar ratio of 0.5:0.5:1:1, and hybridization was conducted;
a preparation method of the BSA&BSA-AgP@gel (encapsulated polypeptide vaccine) included: the BSA and the prepared BSA-AgP were mixed with linker DNA and a Y-shaped scaffold, where the BSA, BSA-AgP, linker DNA, and the Y-shaped scaffold were at a molar ratio of 0.5:0.5:1:1;
a preparation method of the (BSA&BSA-AgP)-gel (full hybrid polypeptide vaccine) included: the BSA linked to GOD-linker-N₃ and BSA-AgP linked to GOD-linker-N₃ were mixed with linker DNA and Y-shaped scaffold, where the BSA, BSA-AgP, linker DNA, and Y-shaped scaffold were at a molar ratio of 0.5:0.5:1:1, and hybridization was conducted.
20 female C57 mice were randomly divided into 5 groups, one of which served as a blank control. The other 4 groups were named an experimental group (BSA@BSA-AgP-gel), an encapsulated group (BSA&BSA-AgP@gel), a full hybrid group ((BSA&BSA-AgP)-gel), and a two-dose group (Free BSA&BSA-AgP). On day 0, the following vaccines were injected subcutaneously into the back of C57 mice. In the experimental group (BSA@BSA-AgP-gel), 20 μg BSA-DNA-S3 and 20 μg BSA-DNA-S4 were hybridized into the DNA hydrogel, and 200 μg BSA was encapsulated into the DNA hydrogel. In the encapsulated group (BSA&BSA-AgP@gel), 20 μg BSA-S3, 20 μg BSA-S4, and 200 μg BSA were encapsulated into the DNA hydrogel. In the full hybrid group ((BSA&BSA-AgP)-gel), 20 μg of BSA-DNA-S3, 20 μg of BSA-DNA-S4, and 200 μg of BSA-DNA were hybridized to the DNA hydrogel. In the two-dose group (Free BSA&BSA-AgP), 20 μg BSA-S3, 20 μg BSA-S4, 200 μg BSA, and 50 μg CpG (the same dose as the CpG contained in the ALG-DNA hydrogel) were injected subcutaneously into the back of C57 mice twice on day 0 and day 7. The immunization timeline was shown in FIG. 6A.

Blood samples were collected from each group on the 14th and 21st d after immunization to determine the specific antibody titer for RBD in mouse serum, and the plates were coated with 1 μg/100 μL of RBD and incubated overnight at 4° C. on a high-absorption 96-well Costar plate. The titer of RBD-specific antibodies on SARS-CoV-2 in the serum of each group of mice was then measured.

14 d after hydrogel vaccination, peripheral blood of mice was collected to measure antibody titers, and the results were shown in FIG. 6B. The experimental results showed that the antibody titer of the encapsulated group (BSA&BSA-AgP@gel) was the lowest among all immunization groups, and there was a significant difference with other groups, indicating that the rapid release of vaccine components could not produce a desirable immune effect, which was consistent with the conclusion of immune exploration in Chapter Two. At this time, the experimental group (BSA@BSA-AgP-gel) produced the highest antibody titer, which was significantly different from that of the full hybrid group ((BSA&BSA-AgP)-gel), indicating that the kinetic differential release vaccine that rapidly released the free carrier protein to activate T cells and then slowly released the peptide vaccine to activate B cells had a better immune effect than that of the vaccine with slow release of all vaccine components. Moreover, the experimental group (BSA@BSA-AgP-gel) also had significant differences from the two-dose group (Free BSA&BSA-AgP), indicating that the humoral immune effect of the kinetic differential release vaccine was better than that produced by the vaccine with two immunizations, which meant that the kinetic differential release vaccine could reduce the number of immunizations.

21 d after hydrogel vaccination, peripheral blood of mice was collected to measure antibody titers, and the results were shown in FIG. 6C. At this time, the differences between the immune effects of the vaccines in each group were similar to those 14 d after vaccination. The antibody titer levels of each immunization group were slightly lower than that of 14 d after immunization, while the experimental group (BSA@BSA-AgP-gel) still maintained a relatively high antibody titer level, indicating that the kinetic differential release vaccine had a relatively long-lasting immune effect.

### Example 8

### Influence of Kinetic Differential Release Based on DNA Hydrogel on CD4⁺ T Cell Activation Induced by Peptide Vaccine

In order to verify the results of T cell proliferation after mice were immunized with polypeptide vaccines constructed with DNA hydrogel, the T cell activation status of different immunization groups at different time points was obtained. Blood was collected from each group of mice described in 5 at 7 d and 14 d after immunization to determine the activation of Th2 CD4⁺ T cells. After blood was collected, anticoagulants and cytokine inhibitors were added and reacted for 1 h. 1.5 mL of red blood cell lysis buffer was added and lysed for about 5 min until the blood became clear and was free of blood clots. Centrifugation was conducted at 1,500 rpm for 3 min and a supernatant was discarded. 1×PBS was added to resuspend and separate some cells for single staining. 0.3 μL FITC-CD3F and 0.3 μL PerCP-CD4 were added to allow staining for 30 min. 300 μL PBS was added and mixed well, then centrifuged at 1,500 rpm for 3 min. A supernatant was discarded and 100 μL of fixative was added to allow fixation for 20 min. 500 μL of 1× membrane permeabilization buffer was added, mixed well, centrifuged at 1,500 r for 3 min, a supernatant was discarded, and the above operations were repeated once. 200 μL of permeabilization buffer was added, resuspended, added with 0.3 μL of PE/Cyanine7 IL-4, and stained for 1 h. 300 μL PBS was added, centrifuged at 1,500 r for 3 min, a supernatant was discarded, 200 μL PBS was added and mixed well to obtain a flow cytometry sample. The proportion of activated Th2 CD4⁺ T cells (IL-4⁺ in CD3⁺CD4⁺) was detected by flow cytometry.

The results showed that the mice in experimental group (BSA@BSA-AgP-gel) had the highest antibody titer 14 d and 21 d after immunization in each group, indicating that the experimental group produced the best humoral immune effect.

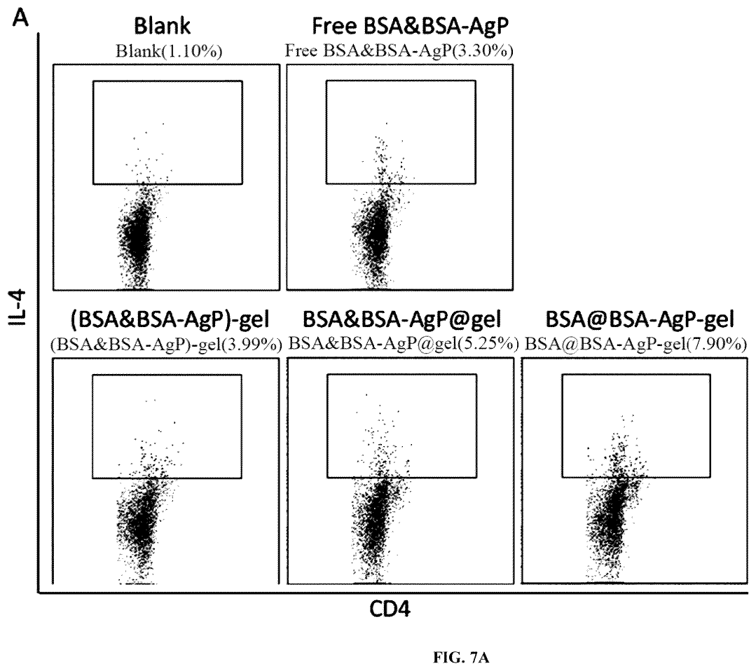

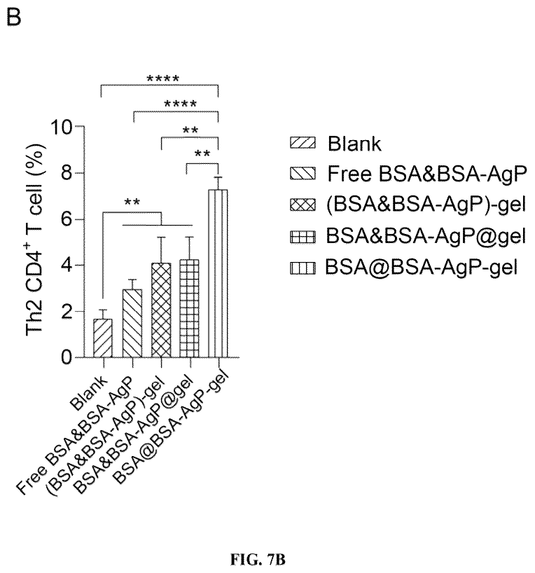

In order to further explore the results of T cell activation in each immune group of mice 7 d and 14 d after immunization, the activation effect of Th2 CD4⁺ T cells in the peripheral blood of mice was measured. The activation ratio of Th2 CD4⁺ T cells in each immune group 7 d after immunization was shown in FIGS. 7A-7B, indicating that each immune group had significant differences compared with the blank group, proving that each immune group had T cell activation one week after immunization. At this time, the T cell activation effect of the two-dose group (Free BSA&BSA-AgP) was the worst, which might be because the two-dose group (Free BSA&BSA-AgP) only used half the dose of the vaccine components of other groups to immunize mice 1 week after immunization. There was no significant difference between the full hybrid group ((BSA&BSA-AgP)-gel) and the encapsulated group (BSA&BSA-AgP@gel). This might be because all vaccine components in the encapsulated group (BSA&BSA-AgP@gel) were quickly released at this time, while some vaccine components in the full hybrid group ((BSA&BSA-AgP)-gel) had not yet been released. The experimental group (BSA@BSA-AgP-gel) had the highest Th2 CD4⁺ T cell activation effect, which was significantly different from that of all other groups, indicating that the kinetic differential release vaccine had a better T cell activation effect than that of the hydrogel immunization groups.

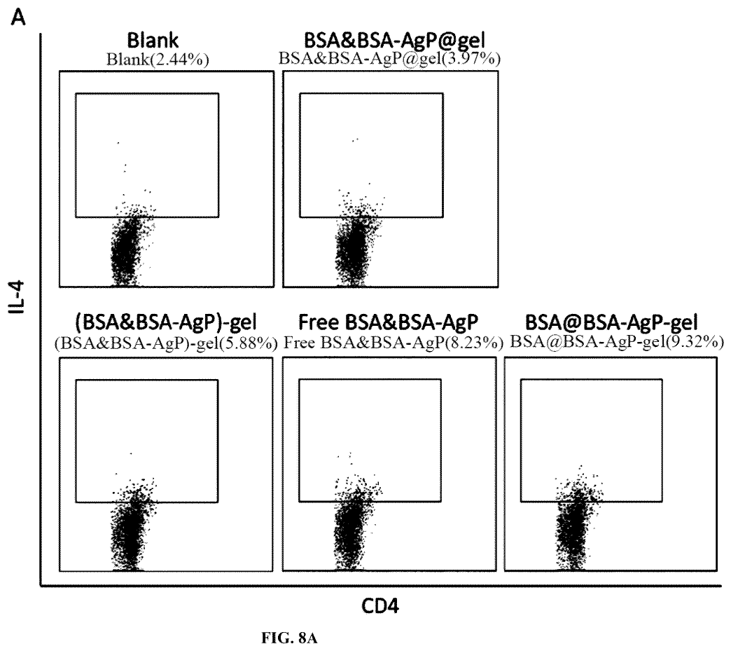

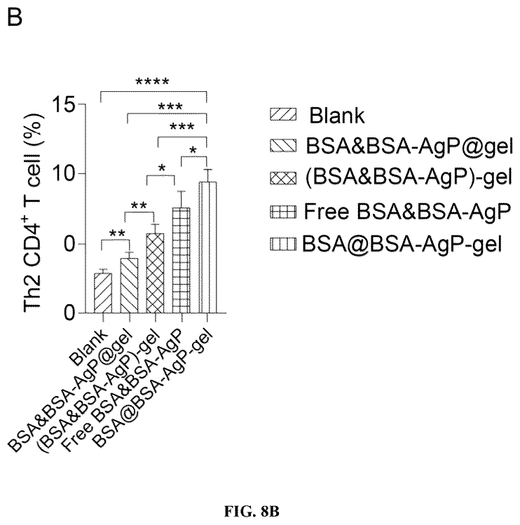

The activation ratio of Th2 CD4⁺ T cells in the peripheral blood of mice in each immunization group 14 d after immunization was shown in FIGS. 8A-8B. The results showed that the proportion of Th2 CD4⁺ T cells in each immunization group was significantly different from that in the blank group 14 d after immunization, indicating that each immunization group still had T cell activation at this time. The encapsulated group (BSA&BSA-AgP@gel) had the lowest T cell activation level, and there was a significant difference between the full hybrid group ((BSA&BSA-AgP)-gel) and the encapsulated group (BSA&BSA-AgP@gel), indicating that the sustained-release vaccine had a better T cell activation effect than that of the burst-release vaccine. The experimental group (BSA@BSA-AgP-gel) still had the highest T cell activation effect 14 d after immunization, and had significant differences among the hydrogel groups, indicating that kinetic differential release had better Th2 CD4⁺ T cell activation effect than that of all sustained release or all burst release. Moreover, the experimental group (BSA@BSA-AgP-gel) also had significant differences compared to the two-dose group (Free BSA&BSA-AgP), indicating that the kinetic differential release vaccine could produce better T cell activation effect than that of two immunizations. The experimental results were consistent with the humoral immunity results described in 6, indicating that T cell activation might affect the humoral immunity effect.

### Example 9

Biosafety Evaluation of Polypeptide Vaccine with Kinetic Differential Release Based on DNA Hydrogel

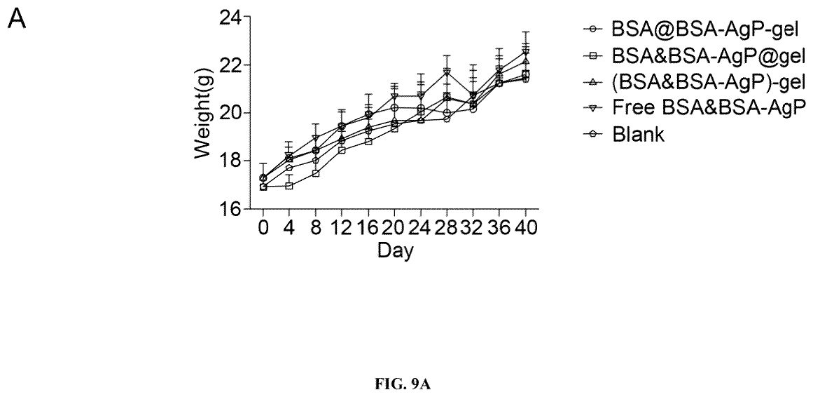

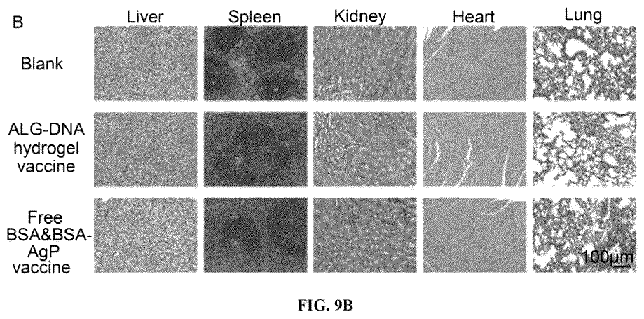

In order to show that the DNA hydrogel vaccine had no toxic effects on mice, the body weight of mice in each group in 5 was weighed every 4 d, and the body weight measured on the day of immunization was the body weight on day 0. The average body weight of mice in each group was calculated, and a line graph was drawn with time as the horizontal axis and body weight as the vertical axis. As shown in FIG. 9A, the weight of the mice did not decrease due to vaccination, but rather increased in a fluctuating manner overall. The weight of mice was counted until the day 40, when the average weight of mice in each group had reached more than 21 g. Moreover, the weight of mice in each immunization group was comparable to that of mice in the blank group, indicating that ALG-DNA hydrogel microspheres as a vaccine carrier did not affect the weight of mice, such that the ALG-DNA hydrogel microspheres had basically no toxic effect on mice.

At the end of the study, mice in the representative blank group, experimental group (BSA@BSA-AgP-gel), and two-dose group (Free BSA&BSA-AgP) were sacrificed, and liver, spleen, kidney, heart, and lung of the mice were dissected out, rinsed with 1×PBS and fixated with 4% paraformaldehyde for 48 h. The fixated mouse organs were embedded in paraffin and cut into sections. After the sections were stained with hematoxylin and eosin (H&E), the tissues were analyzed by microscopy. As shown in FIG. 9B, there was no damage to the tissues of mice in each group, the cell nuclei were clear, the contrast between the nuclei and cytoplasm was sharp, and no lesions were observed, indicating that the hydrogel immunization or direct injection of vaccine components in each group of mice would not cause toxicity to the body, proving that the hydrogel vaccine was biosafe.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A DNA adjuvant hydrogel-based peptide vaccine, comprising a peptide-carrier protein, a DNA hydrogel, and a free carrier protein; wherein
the peptide-carrier protein is loaded in the DNA hydrogel or encapsulated in the DNA hydrogel by hybridization;
the free carrier protein is encapsulated inside the DNA hydrogel; and
the DNA hydrogel is self-assembled from a Y-shaped scaffold and a linker DNA.

2. The DNA adjuvant hydrogel-based peptide vaccine according to claim 1, wherein the Y-shaped scaffold is assembled from DNA strands having nucleotide sequences shown in SEQ ID NO: 1 to SEQ ID NO: 3; and
the linker DNA is assembled from DNA strands having nucleotide sequences shown in SEQ ID NO: 4 to SEQ ID NO: 5.

3. The DNA adjuvant hydrogel-based peptide vaccine according to claim 1, wherein a quantity of the Y-shaped scaffolds and a quantity of the linker DNAs are at a ratio of 1:1; and
the DNA hydrogel has a molar concentration of 200 μM to 300 μM.

4. The DNA adjuvant hydrogel-based peptide vaccine according to claim 1, wherein a preparation process of the DNA hydrogel comprises the following steps:
mixing DNA strands having nucleotide sequences shown in SEQ ID NO: 1 to SEQ ID NO: 3 or DNA strands having nucleotide sequences shown in SEQ ID NO: 4 to SEQ ID NO: 5 at an equal molar ratio in a buffer, and then treating at 95° C. for 15 min to obtain the Y-shaped scaffold and the linker DNA; and
mixing the Y-shaped scaffold and the linker DNA to obtain the DNA hydrogel.

5. The DNA adjuvant hydrogel-based peptide vaccine according to claim 1, wherein a peptide in the peptide-carrier protein comprises a B cell epitope polypeptide of a pathogen protein antigen;
preferably, the B cell epitope polypeptide of the pathogen protein antigen comprises S3 and S4 of a receptor-binding domain (RBD) of an S protein of a severe acute respiratory syndrome coronavirus 2 (SARS-CoV-2); and
preferably, a carrier protein in the peptide-carrier protein is at least one selected from the group consisting of BSA, OVA, and KLH.

6. The DNA adjuvant hydrogel-based peptide vaccine according to claim 1, wherein a preparation process of the peptide-carrier protein comprises the following steps: subjecting a carrier protein BSA to a shaking reaction with dibenzocyclooctyne-N-hydroxysuccinimide (DBCO-NHS), removing unreacted components, and then adding a peptide into an obtained reaction product to allow a freezing reaction; wherein
the BSA has a molar concentration of 100 μM to 102 μM;
the DBCO-NHS is added at a molar concentration 28 to 32 times the molar concentration of the BSA; and
the peptide is added at a molar concentration 5 to 15 times the molar concentration of the BSA.

7. The DNA adjuvant hydrogel-based peptide vaccine according to claim 1, wherein the peptide-carrier protein is covalently linked to GOD-linker-N₃ and hybridized with DNA strands in the DNA hydrogel; and
the GOD-linker-N₃ has a nucleotide sequence shown in SEQ ID NO: 6.

8. The DNA adjuvant hydrogel-based peptide vaccine according to claim 7, wherein a preparation process of a GOD-linker-N₃-linked peptide-carrier protein comprises: adding the GOD-linker-N₃ while adding the peptide at a molar concentration 5 to 15 times the molar concentration of the BSA into the reaction product when the peptide-carrier protein is prepared; wherein
the GOD-linker-N₃ is added at 1 to 3 times the molar concentration of the BSA.

9. A preparation method of the DNA adjuvant hydrogel-based peptide vaccine according to claim 1, comprising a preparation process of a DNA adjuvant hydrogel-based peptide vaccine encapsulating the free carrier protein and the peptide-carrier protein and a preparation process of a DNA adjuvant hydrogel-based peptide vaccine encapsulating the free carrier protein and a hybrid peptide-carrier protein; wherein
the preparation process of the DNA adjuvant hydrogel-based peptide vaccine encapsulating the free carrier protein and the peptide-carrier protein comprises: mixing the free carrier protein and the peptide-carrier protein with the linker DNA, and then mixing an obtained mixture with the Y-shaped scaffold; and
the preparation process of the DNA adjuvant hydrogel-based peptide vaccine encapsulating the free carrier protein and the hybrid peptide-carrier protein comprises: mixing the free carrier protein and the GOD-linker-N₃-linked peptide-carrier protein with the linker DNA, and then mixing an obtained mixture with the Y-shaped scaffold.

10. The DNA adjuvant hydrogel-based peptide vaccine according to claim 6, wherein the peptide-carrier protein is covalently linked to GOD-linker-N₃ and hybridized with DNA strands in the DNA hydrogel; and
the GOD-linker-N₃ has a nucleotide sequence shown in SEQ ID NO: 6.

11. The DNA adjuvant hydrogel-based peptide vaccine according to claim 10, wherein a preparation process of a GOD-linker-N₃-linked peptide-carrier protein comprises: adding the GOD-linker-N₃ while adding the peptide at a molar concentration 5 to 15 times the molar concentration of the BSA into the reaction product when the peptide-carrier protein is prepared; wherein
the GOD-linker-N₃ is added at 1 to 3 times the molar concentration of the BSA.

12. The preparation method according to claim 9, wherein the Y-shaped scaffold is assembled from DNA strands having nucleotide sequences shown in SEQ ID NO: 1 to SEQ ID NO: 3; and
the linker DNA is assembled from DNA strands having nucleotide sequences shown in SEQ ID NO: 4 to SEQ ID NO: 5.

13. The preparation method according to claim 9, wherein a quantity of the Y-shaped scaffolds and a quantity of the linker DNAs are at a ratio of 1:1; and
the DNA hydrogel has a molar concentration of 200 μM to 300 μM.

14. The preparation method according to claim 9, wherein a preparation process of the DNA hydrogel comprises the following steps:
mixing DNA strands having nucleotide sequences shown in SEQ ID NO: 1 to SEQ ID NO: 3 or DNA strands having nucleotide sequences shown in SEQ ID NO: 4 to SEQ ID NO: 5 at an equal molar ratio in a buffer, and then treating at 95° C. for 15 min to obtain the Y-shaped scaffold and the linker DNA; and
mixing the Y-shaped scaffold and the linker DNA to obtain the DNA hydrogel.

15. The preparation method according to claim 9, wherein a peptide in the peptide-carrier protein comprises a B cell epitope polypeptide of a pathogen protein antigen;
preferably, the B cell epitope polypeptide of the pathogen protein antigen comprises S3 and S4 of a receptor-binding domain (RBD) of an S protein of a severe acute respiratory syndrome coronavirus 2 (SARS-CoV-2); and
preferably, a carrier protein in the peptide-carrier protein is at least one selected from the group consisting of BSA, OVA, and KLH.

16. The preparation method according to claim 9, wherein a preparation process of the peptide-carrier protein comprises the following steps: subjecting a carrier protein BSA to a shaking reaction with dibenzocyclooctyne-N-hydroxysuccinimide (DBCO-NHS), removing unreacted components, and then adding a peptide into an obtained reaction product to allow a freezing reaction; wherein
the BSA has a molar concentration of 100 μM to 102 μM;
the DBCO-NHS is added at a molar concentration 28 to 32 times the molar concentration of the BSA; and
the peptide is added at a molar concentration 5 to 15 times the molar concentration of the BSA.

17. The preparation method according to claim 9, wherein the peptide-carrier protein is covalently linked to GOD-linker-N₃ and hybridized with DNA strands in the DNA hydrogel; and
the GOD-linker-N₃ has a nucleotide sequence shown in SEQ ID NO: 6.

18. The preparation method according to claim 17, wherein a preparation process of a GOD-linker-N₃-linked peptide-carrier protein comprises: adding the GOD-linker-N₃ while adding the peptide at a molar concentration 5 to 15 times the molar concentration of the BSA into the reaction product when the peptide-carrier protein is prepared; wherein
the GOD-linker-N₃ is added at 1 to 3 times the molar concentration of the BSA.


SEQUENCE LISTING (8 sequences)

SEQ ID NO: 1 - DNA, 32 bp, synthetic construct
  source 1..32: mol_type = other DNA; note = 2006-1-S
cgattgactcgtcgttttgtcgttttgtcgtt

SEQ ID NO: 2 - DNA, 32 bp, synthetic construct
  source 1..32: mol_type = other DNA; note = 2006-2
cgattgacaacgacaaaacgcacgctgtccta

SEQ ID NO: 3 - DNA, 32 bp, synthetic construct
  source 1..32: mol_type = other DNA; note = 2006-3
cgattgactaggacagcgtgacaaaacgacga

SEQ ID NO: 4 - DNA, 49 bp, synthetic construct
  source 1..49: mol_type = other DNA; note = 1826-1-linker-S
gtgcttggtaacacagctcctgtcaatcgtccatgacgttcctgacgtt

SEQ ID NO: 5 - DNA, 28 bp, synthetic construct
  source 1..28: mol_type = other DNA; note = 1826-2
gtcaatcgaacgtcaggaacgtcatgga

SEQ ID NO: 6 - DNA, 20 bp, synthetic construct
  source 1..20: mol_type = other DNA; note = GOD-linker-N3
ggagctgtgttaccaagcac

SEQ ID NO: 7 - protein, 9 aa, synthetic construct
  source 1..9: note = Amino acid sequence of S3
GDEVRQIAP

SEQ ID NO: 8 - protein, 6 aa, synthetic construct
  source 1..6: note = Amino acid sequence of S4
NLDSKV